(19) United States
(12) Patent Application Publication
(10) Pub. No.: US 2012/0116357 A1
(43) Pub. Date: May 10, 2012

(54) URINARY CATHETER STABILIZER AND METHOD OF USE

(76) Inventors: SAID I. HAKKY, LARGO, FL (US); SHERAZHAD S. HAKKY, LARGO, FL (US); SHEREEN S. HAKKY, LARGO, FL (US)

(21) Appl. No.: 12/948,148

(22) Filed: Nov. 17, 2010

Related U.S. Application Data
(60) Provisional application No. 61/411,670, filed on Nov. 9, 2010.

(51) Int. Cl. A61M 27/00

(52) U.S. Cl. 604/544

(57) ABSTRACT

A urinary catheter stabilizer comprises a body member, a rotatable adhesion member, and an adhesive laminar membrane. The body member includes an upper cradle member that is hingedly attached to a lower cradle member. Each of the upper and lower cradle members is adapted to receive a bellows of a urinary catheter. The bellows includes oppositely disposed proximal and distal ends and a ribbed portion extending therebetween. Each of the upper and lower cradle members includes a proximal end portion, a distal end portion, and a main body portion extending between the proximal and distal end portions. The rotatable adhesion member is securely connected to the lower cradle member. The rotatable adhesion member includes a base portion having oppositely disposed first and second surfaces. The adhesive laminar membrane includes oppositely disposed first and second major surfaces. A portion of the first major surface is securely attached to the first surface of the base portion of the rotatable adhesion member.

### RELATED APPLICATION

This application claims priority from U.S. Provisional Application No. 61/411,670, filed Nov. 9, 2010, the entirety of which is hereby incorporated by reference.

### TECHNICAL FIELD

The present invention relates generally to an apparatus and method for stabilizing a catheter, and more particularly to an apparatus and method for stabilizing a urinary catheter having at least one bellows.

### BACKGROUND OF THE INVENTION

Devices for stabilizing urethral catheter are required to perform two primary functions. First, the device must hold the catheter tube positionally stabilized relative to the device itself. Second, the device itself must be held positionally stabilized relative to the body of the patient. Prior art urethral catheter holding devices generally fail to satisfactorily perform one or both of these functions. For example, some devices require conventional adhesive tape to be secured to the patient's skin; such tape, as a general rule, does not hold in place for any considerable period of time and is often time-consuming and bothersome to employ. Other devices utilize stretch rubber strips with VELCRO fastening devices, wherein the strips encircle the patient's thigh. These are unsatisfactory because the strips must be wrapped so tightly as to constrict blood circulation to prevent the device from slipping along the thigh. Other devices require that the tube be tied, taped, or similarly engaged to the device; however, such arrangements tend to restrict the flow through the catheter tube. Finally, there are other devices which require special configurations of catheter tubes to engage the tube properly.

Such custom tubing requirements are undesirable as they are not useful for the majority of commercially-available catheters.

### SUMMARY OF THE INVENTION

According to one aspect of the present invention, a urinary catheter stabilizer comprises a body member, a rotatable member, and an adhesive laminar membrane. The body member includes an upper cradle member that is hingedly attached to a lower cradle member. Each of the upper and lower cradle members is adapted to receive a bellows of a urinary catheter. The bellows includes oppositely disposed proximal and distal ends and a ribbed portion extending therebetween. Each of the upper and lower cradle members includes a proximal end portion, a distal end portion, and a main body portion extending between the proximal and distal end portions. The rotatable member is securely connected to the lower cradle member. The rotatable adhesion member includes a base portion having oppositely disposed first and second surfaces. The adhesive laminar membrane includes oppositely disposed first and second major surfaces. A portion of the first major surface is securely attached to the first surface of the base portion of the rotatable member.

According to another aspect of the present invention, a urinary catheter stabilizer comprises a body member, a rotatable member, and an adhesive laminar membrane. The body member includes an upper cradle member that is hingedly attached to a lower cradle member. Each of the upper and lower cradle members is adapted to receive a bellows of a urinary catheter. The bellows includes oppositely disposed proximal and distal ends and a ribbed portion extending therebetween. Each of the upper and lower cradle members includes a proximal end portion, a distal end portion, and a main body portion extending between the proximal and distal end portions. The body member includes a plurality of locking mechanisms for securely mating the upper and lower cradle members. Each of the first and second end portions of each of the upper and lower cradle members is adapted to securely receive the proximal and distal ends of the bellows, respectively, and thereby prevent or mitigate constriction of the urinary catheter. The rotatable member is securely connected to the lower cradle member. The rotatable adhesion member includes a base portion having oppositely disposed first and second surfaces. The adhesive laminar membrane includes oppositely disposed first and second major surfaces. A portion of the first major surface is securely attached to the first surface of the base portion of the rotatable member.

According to another aspect of the present invention, a urinary catheter stabilizer comprises a body member, a rotatable member, and an adhesive laminar membrane. The body member includes an upper cradle member that is hingedly attached to a lower cradle member. Each of the upper and lower cradle members is made of a transparent or semi-transparent material and is adapted to receive a bellows of a urinary catheter. The bellows includes oppositely disposed proximal and distal ends and a ribbed portion extending therebetween. Each of the upper and lower cradle members includes a proximal end portion, a distal end portion, and a main body portion extending between the proximal and distal end portions. The body member includes a plurality of locking mechanisms for securely mating the upper and lower cradle members. Each of the first and second end portions of each of the upper and lower cradle members is adapted to securely receive the proximal and distal ends of the bellows, respectively, and thereby prevent or mitigate constriction of the urinary catheter. The rotatable member is securely connected to the lower cradle member. The rotatable adhesion member includes a base portion having oppositely disposed first and second surfaces. The adhesive laminar membrane includes oppositely disposed first and second major surfaces. The adhesive laminar membrane is made of a transparent or semi-transparent material and includes a water-resistant, hypoallergenic adhesive disposed on at least a portion of the second major surface. A portion of the first major surface is securely attached to the first surface of the base portion of the rotatable member.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view showing a urinary catheter stabilizer in an open configuration and constructed in accordance with one aspect of the present invention;

FIG. 2 is a top view of the urinary catheter stabilizer in FIG. 1 in a closed configuration and seated on an adhesive laminar membrane;

FIG. 3 is a perspective view of the urinary catheter stabilizer in FIG. 2;

FIG. 4 is a perspective view showing a body member of the urinary catheter stabilizer in FIG. 1;

FIG. 5 is an alternative perspective view of the body member in FIG. 4;

FIG. 6 is a side view of the body member in FIG. 5;

FIG. 7 is an alternative perspective view of the body member in FIG. 6;

FIG. 8 is a perspective view showing a rotatable member of the urinary catheter stabilizer in FIG. 1;

FIG. 9 is an alternative perspective view of the rotatable member in FIG. 8;

FIG. 10 is a perspective view showing an adhesive laminar membrane of the urinary catheter stabilizer in FIG. 1; and

FIG. 11 is an alternative perspective view of the adhesive laminar member in FIG. 10.

### DETAILED DESCRIPTION

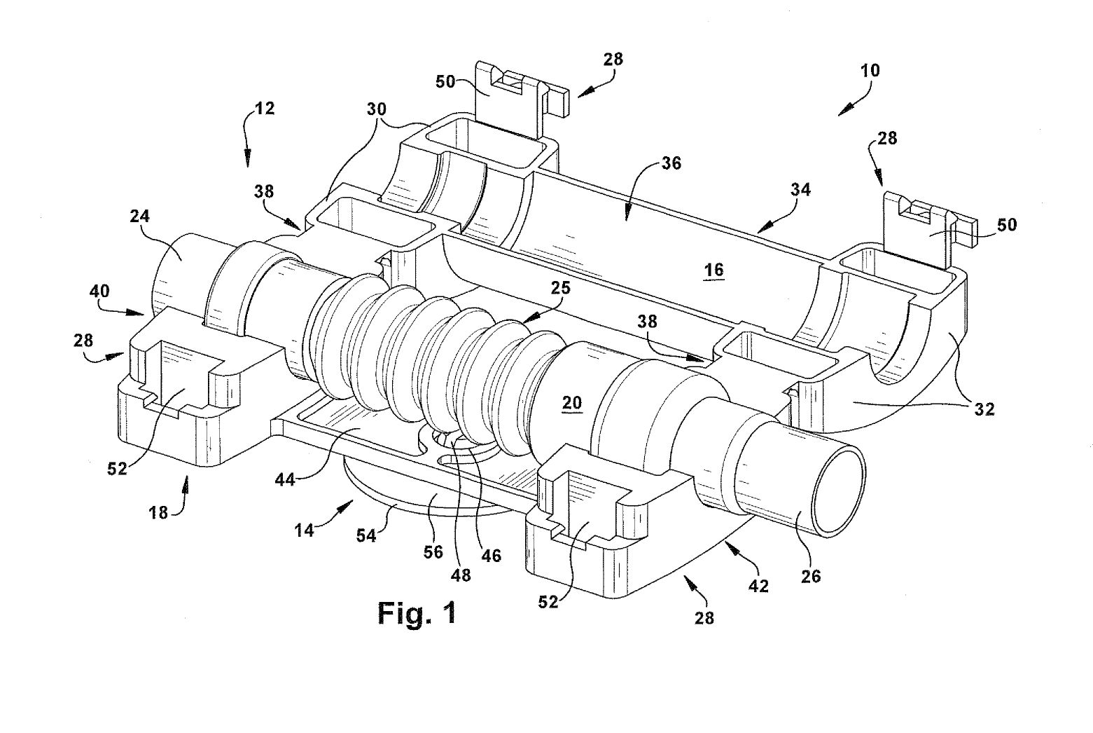

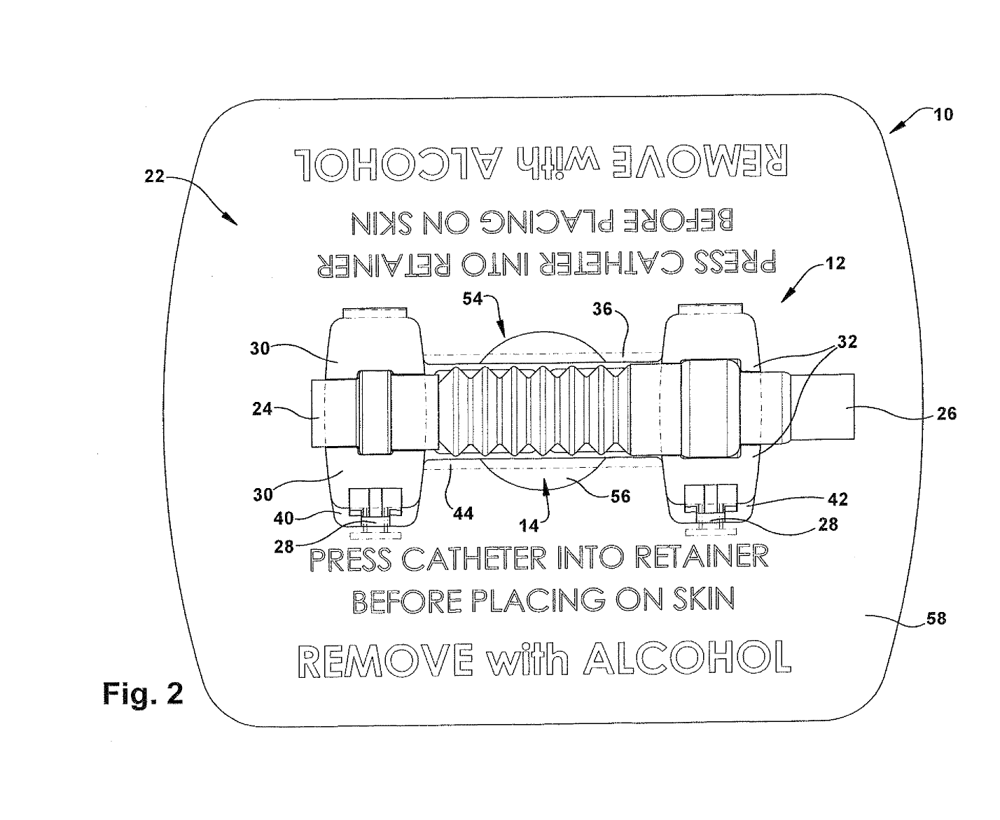

The present invention relates generally to an apparatus and method for stabilizing a catheter, and more particularly to an apparatus and method for stabilizing a urinary catheter having at least one bellows. As representative of one aspect of the present invention, FIGS. 1-3 illustrate a disposable urinary catheter stabilizer 10 that conveniently adheres to a patient and avoids dislodgement of a urinary catheter (not shown) from the patient. As shown in FIG. 1, the urinary catheter stabilizer 10 comprises a body member 12 that is rotatably connected to a rotatable member 14. The body member 12 comprises upper and lower cradle members 16 and 18 that are hingedly connected to one another and adapted to receive a bellows 20 of urinary catheter (or other similar instrument). As discussed in more detail below, the urinary catheter stabilizer 10 also includes an adhesive laminar membrane 22 (FIG. 2) that is securely attached to the rotatable member 14.

Although devices for stabilizing urinary catheters are known in the art, their particular designs are disadvantageous for several reasons. The STATLOCK Foley Stabilization Device (Bard Medical Division, Covington, Ga.), for example, secures the device to the patient with an adhesive pad, which provides a sanitary safeguard that prevents patient or item interference. The STATLOCK's releasable single lock retainer swivels to enhance user comfort, while the adhesive pad permits fast application. Nevertheless, the lock retainer may constrict larger French size catheters and the STATLOCK's adhesive may elicit skin reactions. The VELCRO method of securing urinary catheters is comparably less expensive, simple, and requires no risk of skin allergy. However, it is difficult to maintain the cleanliness of VELCRO, which can also potentially constrict the catheter if the patient or caregiver attaches the straps too firmly. Additionally, irrigation of urinary catheters is currently needed to clear blood clots or debris. When using both the STATLOCK and the VELCRO straps, the catheter must be freed first and, only after irrigation is concluded, can the catheter stabilizer be reapplied.

Unlike the stabilization devices of the prior art, the urinary catheter stabilizer 10 of the present invention provides superior support for urinary catheters as the surface area of the stabilizer is comparably greater than the prior art devices, as well as greater catheter security via the upper and lower cradle members 16 and 18. The urinary catheter stabilizer 10 also secures the proximal and distal ends 24 and 26 of the bellows 20, which provides a semi-rigid surface such that the likelihood of catheter obstruction—a primary concern during bladder irrigation—is obsolete. Additionally, at least a portion of the body member 12 (FIGS. 1-3) of the present invention is manufactured with a transparent or semi-transparent material (e.g., plastic) so that interference or malfunction can be readily identified and sanitation of the stabilizer 10 is easily observed. Other advantages of the present invention are described below.

One aspect of the present invention includes a stabilizer 10 for conveniently securing a urinary catheter to a portion of a patient, such as an inner thigh of the patient. The present invention is described below in terms of providing a stabilizer 10 for the bellows 20 of a Hakki urinary catheter, which is disclosed in U.S. patent application Ser. No. 12/490,669 and incorporated herein by reference. It will be appreciated, however, that the present invention can be adapted to accommodate any French gauge catheter and thereby provide ultimate versatility. As described in more detail below, the upper and lower cradle members 16 and 18 are adapted to securely receive proximal and distal ends 24 and 26 of the bellows 20, respectively, and thereby prevent or mitigate catheter constriction. Additionally, the upper and lower cradle members 16 and 18 protect the ribbed portion 25 of the bellows 20 so that the ribbed portion is not deformed during motion of the patient.

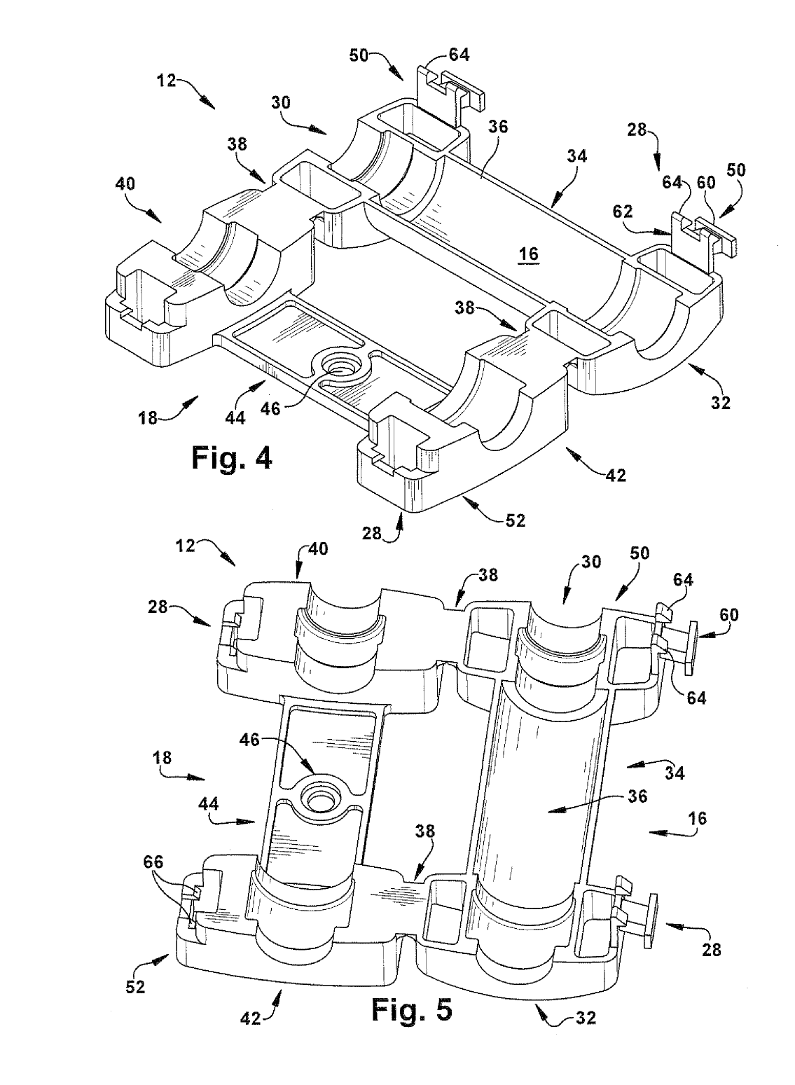

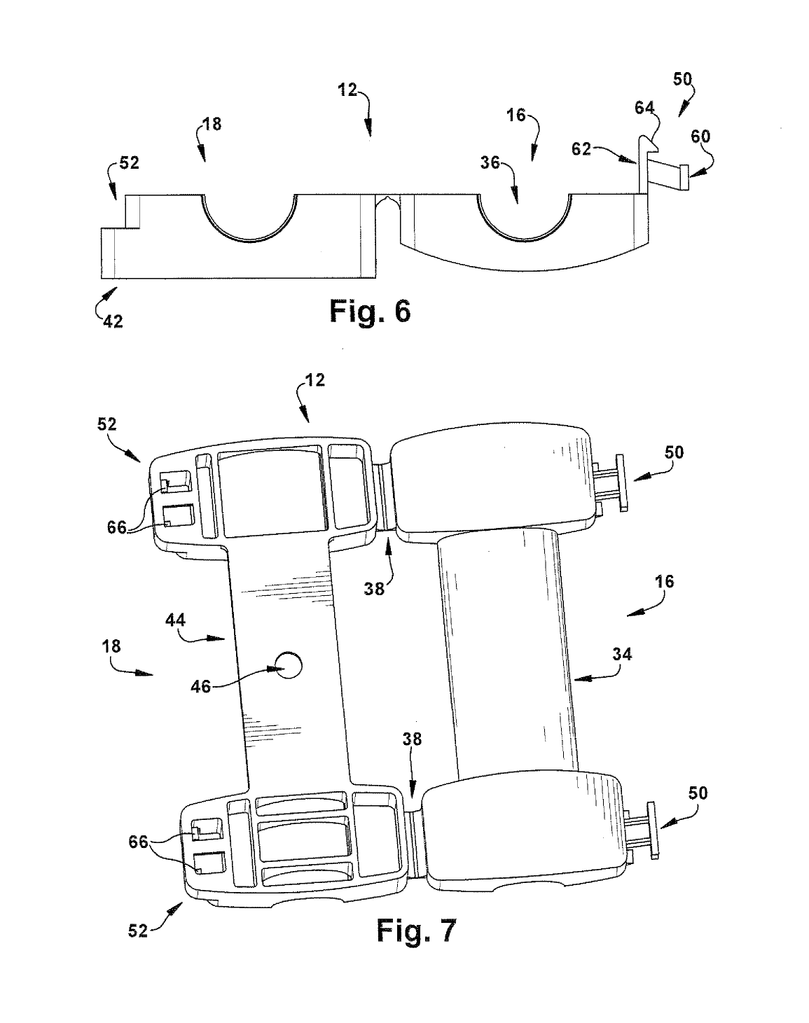

The urinary catheter stabilizer 10 comprises a rigid or semi-rigid body member 12 (FIGS. 4-7) for supporting the bellows 20 of a Hakki urinary catheter. The body member 12 (FIGS. 4-7) includes upper and lower cradle members 16 and 18 that are hingedly connected to one another and capable of being mated together via a plurality of locking mechanisms 28. The upper cradle member 16 functions primarily as a protective cover to guard the bellows 20 (i.e., the ribbed portion 25) from unintentional alteration or damage. The upper cradle member 16 has an elongated configuration and includes a proximal end portion 30, a distal end portion 32, and a main body portion 34 that extends between the proximal and distal end portions. At least a portion of the upper cradle member 16 is made of a transparent material (e.g., plastic) so that any interference or malfunction in the urinary catheter can be readily identified, and the cleanliness of the stabilizer 10 can be readily observed.

The main body portion 34 of the upper cradle member 16 generally defines an arch-shaped bellows engagement portion 36. The bellows engagement portion 36 can directly contact the ribbed portion 25 of the bellows 20, or simply cover the ribbed portion (but without contacting the ribbed portion) when the stabilizer 10 is in the closed configuration. The bellows engagement portion 36 extends between the proximal and distal end portions 30 and 32 of the upper cradle member 16, and is adapted to secure a portion of the bellows 20 therein when the urinary catheter stabilizer 10 is in the closed configuration. As shown in FIGS. 4-5, the distal end portion 32 of the upper cradle member 16 is larger than the proximal end portion 30 to accommodate the larger distal end 26 of the bellows 20.

As shown in FIGS. 4-7, the lower cradle member 18 is connected to the upper cradle member 16 by a plurality of flexible hinges 38. The hinges 38 can be integrally formed with the upper and lower cradle members 16 and 18 or, alternatively, formed from a different material that is separately attached (e.g., integrated into) to each of the cradle members. The lower cradle member 18 has an elongated configuration and includes a proximal end portion 40, a distal end portion 42, and a main body portion 44 that extends between the proximal and distal end portions. The proximal end portion 40 is configured to accommodate the corresponding proximal bellows end 24 of the Hakki urinary catheter, which includes a single fitted latex sleeve over the bellows. Additionally, the distal end portion 42 is larger than the proximal end portion 40 as it is configured to accommodate the double latex sleeve at the distal bellows end 26 of the Hakki urinary catheter. The main body portion 44 of the lower cradle member 18 has a flattened, sheet-like configuration and, as described in more detail below, includes an aperture 46 that is adapted to receive a neck portion 48 of the rotatable member 14. All or only a portion of the lower cradle member 18 can be made of a transparent, semi-transparent, and/or non-transparent material, such as plastic.

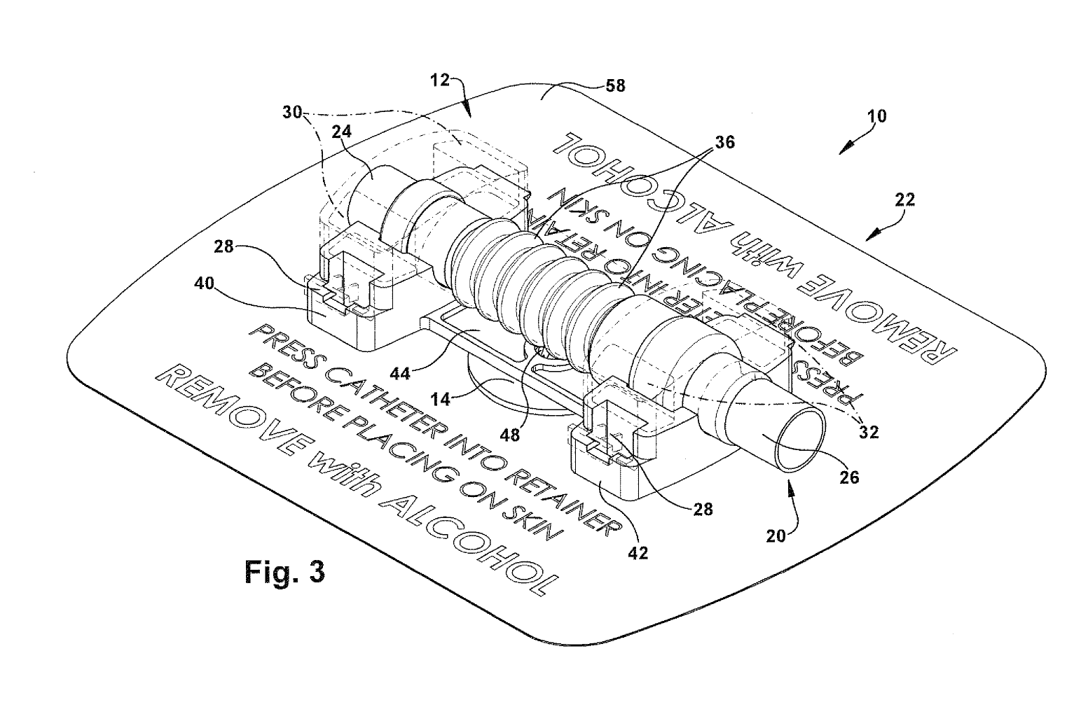

The urinary catheter stabilizer 10 can be moved between an open configuration (FIG. 1) and a closed configuration (FIGS. 2-3) by engaging the locking mechanisms 28, which prevent the obstruction or dislodgement of the urinary catheter (or other similar instrument). As shown in FIGS. 4-6, each of the locking mechanisms 28 has a snap-fit configuration and includes a male end 50 and a female end 52. The male end 50 has a tab-like configuration and is integrally formed with the proximal and distal end portions 30 and 32 of the upper cradle member 16. Each of the male ends 50 includes a release tab 60 that is integrally formed with, and extends generally axial to, an engagement portion 62. The engagement portion 62 includes teeth members 64 that can be friction fit within each of the female ends 52 when the stabilizer 10 is in the closed configuration.

The female end 52 comprises a recessed portion or cavity located within the proximal and distal end portions 40 and 42 of the lower cradle member 18. The cavity of each female end 52 includes at least two ridges 64 (FIG. 5 and FIG. 7) for engaging the teeth members 64 of each male end 50. In operation, the bellows 20 of a Hakki urinary catheter is first seated within the lower cradle member 18. The upper cradle member 16 is then mated with the lower cradle member 18 such that the male end 50 of each of the locking mechanisms 28 engages each of the female ends 52 and thereby securely snap-fits the upper and lower cradle members together. To release the upper cradle member 16, pressure is applied to each of the release tabs 60 while the upper cradle member is simultaneously lifted and disengaged from the lower cradle member 18.

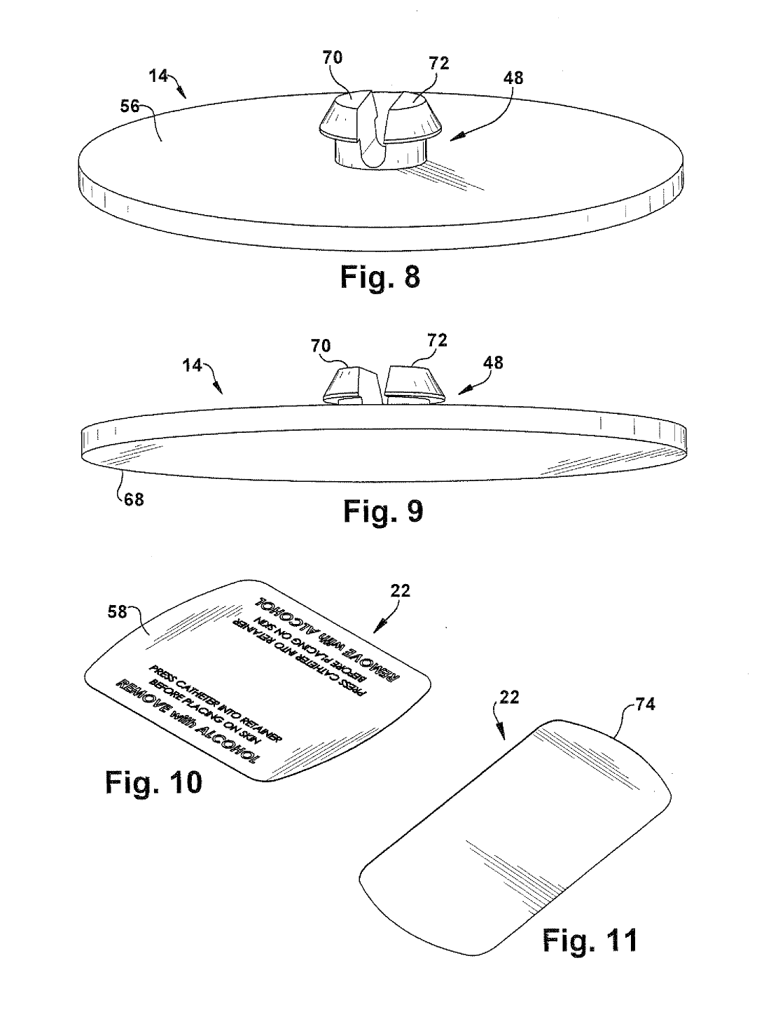

The urinary catheter stabilizer 10 (FIGS. 1-3) additionally includes a rotatable member 14 that is securely connected to the lower cradle member 18. As shown in FIGS. 8-9, the rotatable member 14 comprises a base portion 54 that is integrally formed with a neck portion 48. The base portion 54 includes a first surface 56 that is oppositely disposed from a second surface 68 (FIG. 9). Although the base portion 54 (FIG. 8) has a circular shape in FIGS. 8-9, it will be appreciated that the base portion can have any desired shape. The neck portion 48 is integrally formed with the first surface 56 of the base portion 54 and extends axially thereto. The neck portion 48 is adapted to mate with the aperture 46 of the lower cradle member 18. As shown in FIG. 8, for example, the neck portion 48 has a split, T-shaped configuration defined by opposing first and second flexible prongs 70 and 72. The first and second flexible prongs 70 and 72 can be manually depressed to insert the neck portion 48 into the aperture 46, and then released to securely connect the rotatable member 14 with the lower cradle member 18. The snap-fit configuration of the neck portion 48 with the lower cradle member 18 permits a 360-degree rotation of the body member 12 about the neck portion. This “swivel design” allows the body member 12 a free range of motion, which prevents or mitigates the risk of catheter dislodgement or tearing.

As shown in FIGS. 2-3, the urinary catheter stabilizer 10 additionally includes an adhesive laminar membrane 22 for securing the body member 12 to a portion of a patient. The adhesive laminar membrane 22 (FIGS. 10-11) includes a first major surface 58, a portion of which is securely attached to the second surface 68 of the base portion 54 of the rotatable member 14. All or a portion of the first major surface 58 can be attached to the second surface 68 via an adhesive, clips, staples, or any other means known in the art. The adhesive laminar membrane 22 also includes a second major surface 74 having a water-resistant, hypoallergenic adhesive disposed thereon. The adhesive laminar membrane 22 can include a separate sheet or membrane (not shown) that can be peeled away from the second major surface 74 to allow the second major surface to be securely attached to the skin of the patient. The hypoallergenic, water-resistant adhesive allows the patient to shower without fear of dislodging the stabilizer 10 (and thus the urinary catheter). The adhesive laminar membrane 22 can be made of a suitable medical grade material, such as nylon. As shown in FIGS. 9-10, the adhesive laminar membrane 22 can include print for safety and/or instructional purposes.

From the above description of the invention, those skilled in the art will perceive improvements, changes and modifications. For example, although the urinary catheter stabilizer 10 is shown as having a generally rectangular configuration, it will be appreciated that the stabilizer can have any other suitable shape to provide convenient stabilization for urinary catheters. Such improvements, changes, and modifications are within the skill of the art and are intended to be covered by the appended claims.

Having described the invention, the following is claimed:

1. A urinary catheter stabilizer comprising:
a body member including an upper cradle member that is hingedly attached to a lower cradle member, each of said upper and lower cradle members being adapted to receive a bellows of a urinary catheter, the bellows including oppositely disposed proximal and distal ends and a ribbed portion extending therebetween, each of said upper and lower cradle members including a proximal end portion, a distal end portion, and a main body portion extending between said proximal and distal end portions;
a rotatable member securely connected to said lower cradle member, said rotatable member including a base portion having oppositely disposed first and second surfaces; and
an adhesive laminar membrane including oppositely disposed first and second major surfaces, a portion of said first major surface being securely attached to said first surface of said base portion of said rotatable member.

2. The urinary catheter stabilizer of claim 1, wherein each of said upper and lower cradle members is made of a transparent or semi-transparent material.

3. The urinary catheter stabilizer of claim 1, wherein said body member includes a plurality of locking mechanisms for securely mating said upper and lower cradle members.

4. The urinary catheter stabilizer of claim 1, wherein each of said first and second end portions of each of said upper and lower cradle members is adapted to securely receive the proximal and distal ends of the bellows, respectively, and thereby prevent or mitigate constriction of the urinary catheter.

5. The urinary catheter stabilizer of claim 1, wherein said upper and lower cradle members protect the ribbed portion of the bellows so that the ribbed portion is not deformed during motion of a patient.

6. The urinary catheter stabilizer of claim 1, wherein said adhesive laminar membrane is transparent or semi-transparent.

7. The urinary catheter stabilizer of claim 1, wherein said adhesive laminar membrane includes a water-resistant, hypoallergenic adhesive disposed on at least a portion of said second major surface.

8. A urinary catheter stabilizer comprising:
a body member including an upper cradle member that is hingedly attached to a lower cradle member, each of said upper and lower cradle members being adapted to receive a bellows of a urinary catheter, the bellows including oppositely disposed proximal and distal ends and a ribbed portion extending therebetween, each of said upper and lower cradle members including a proximal end portion, a distal end portion, and a main body portion extending between said proximal and distal end portions, each of said first and second end portions of each of said upper and lower cradle members being adapted to securely receive the proximal and distal ends of the bellows, respectively, said body member including a plurality of locking mechanisms for securely mating said upper and lower cradle members;
a rotatable member securely connected to said lower cradle member, said rotatable member including a base portion having oppositely disposed first and second surfaces; and
an adhesive laminar membrane including oppositely disposed first and second major surfaces, a portion of said first major surface being securely attached to said first surface of said base portion of said rotatable member.

9. The urinary catheter stabilizer of claim 8, wherein each of said upper and lower cradle members is made of a transparent or semi-transparent material.

10. The urinary catheter stabilizer of claim 8, wherein said upper and lower cradle members protect the ribbed portion of the bellows so that the ribbed portion is not deformed during motion of a patient.

11. The urinary catheter stabilizer of claim 8, wherein said adhesive laminar membrane is transparent or semi-transparent.

12. The urinary catheter stabilizer of claim 8, wherein said adhesive laminar membrane includes a water-resistant, hypoallergenic adhesive disposed on at least a portion of said second major surface.

13. A urinary catheter stabilizer comprising:
a body member including an upper cradle member that is hingedly attached to a lower cradle member, each of said upper and lower cradle members being adapted to receive a bellows of a urinary catheter, the bellows including oppositely disposed proximal and distal ends and a ribbed portion extending therebetween, each of said upper and lower cradle members including a proximal end portion, a distal end portion, and a main body portion extending between said proximal and distal end portions, each of said first and second end portions of each of said upper and lower cradle members being adapted to securely receive the proximal and distal ends of the bellows, respectively, said body member including a plurality of locking mechanisms for securely mating said upper and lower cradle members;
a rotatable member securely connected to said lower cradle member, said rotatable member including a base portion having oppositely disposed first and second surfaces; and
an adhesive laminar membrane including oppositely disposed first and second major surfaces, a portion of said first major surface being securely attached to said first surface of said base portion of said rotatable member, at least a portion of said second major surface including a water-resistant, hypoallergenic adhesive disposed thereon;
wherein each of said upper cradle member, said lower cradle member, and said adhesive laminar membrane is transparent.
